(19) United States
(12) Patent Application Publication
(10) Pub. No.: US 2013/0094658 A1
(43) Pub. Date: Apr. 18, 2013

(54) SOUND EXPOSURE MONITOR FOR HEARING PROTECTION DEVICE

(71) Applicant: Trym Holter, Trondheim (NO)

(72) Inventor: Trym Holter, Trondheim (NO)

(73) Assignee: Honeywell International Inc., Morristown, NJ (US)

(21) Appl. No.: 13/275,216

(22) Filed: Oct. 17, 2011

(51) Int. Cl. A61F 11/06

(52) U.S. Cl. 381/72

(57) ABSTRACT

A system and method include sensing sound on the inside of a protective seal of a hearing protection device being worn, and using a sliding window algorithm to calculate the sound to which the ear of the wearer is exposed. The method may also include sensing noise and generating sound to cancel the sensed noise within the ear of the wearer. It may also include sensing ambient sound and regenerating this sound at safe levels inside the hearing protection device in order to aid situational awareness, and it may include receiving signals that are then regenerated as audio inside the hearing protection device for communication or entertainment purposes.

### BACKGROUND

In many industrial settings, workers are routinely exposed to potentially damaging noise environments during their workday. The issue of potential hearing damage often arises in manufacturing and other industrial facilities, but may also arise in military settings, airport settings, and other work environments that involve potentially damaging noise exposure.

Health and Safety regulations around the world set permissible exposure limits for a range of environmental factors, including noise. The main tool for assessing personal cumulative noise exposure is the noise dosimeter, or more precisely, the personal sound exposure meter (PSEM). Historically, PSEMs were designed to be body-worn, typically on the chest or shoulder. When a hearing protection device (HPD) is being used, a body-worn PSEM does not give the complete picture as the attenuation performance of the HPD is unknown.

It is a well established fact that HPD attenuation varies a great deal between different users, and in some cases also between work shifts for the same user. For this reason the first PSEMs integrated in HPDs have been released into the market quite recently. These are designed to measure sound exposure on the inside of the HPD, thereby avoiding the need to estimate the HPD attenuation in order to assess personal sound exposure.

### SUMMARY

A system and method include sensing sound within an ear of a wearer of a hearing protection device, and using a sliding window algorithm to calculate noise to which the ear of the wearer is exposed.

The method may also include sensing noise and generating sound to cancel the sensed noise within the ear of the wearer. It may also include sensing ambient sound and regenerating this sound at safe levels inside the hearing protection device in order to aid situational awareness. The method may further include receiving signals that are then regenerated as audio inside the hearing protection device for communication or entertainment purposes.

In one embodiment, the method is incorporated in instructions stored on a computer readable storage device to cause a computer to implement the method.

### BRIEF DESCRIPTION OF THE DRAWINGS

FIG. 1A is a block diagram of a hearing protection device having an integrated sound exposure monitoring according to an example embodiment.

FIG. 1B is a block diagram of an alternative hearing protection device having integrated sound exposure monitoring according to an example embodiment.

FIG. 2 is a block schematic diagram of a sliding window sound exposure method according to an example embodiment.

FIG. 3 is a graph of data illustrating sound exposure as a function of time according to an example embodiment.

FIG. 4 is a graph of data illustrating calculation of sound exposure at a given time for a window according to an example embodiment.

FIGS. 5A and 5B illustrate the sound exposure calculated according to the conventional approach compared to sound exposure calculated by the sliding window method according to an example embodiment.

FIG. 6 illustrates an earpiece with an external electronic unit according to an example embodiment.

FIG. 7 illustrates an all in ear noise protection device having integrated sound exposure monitoring according to an example embodiment.

FIG. 8 is a block schematic diagram of electronics utilized to perform noise protection and exposure monitoring according to an example embodiment.

### DETAILED DESCRIPTION

In the following description, reference is made to the accompanying drawings that form a part hereof, and in which is shown by way of illustration specific embodiments which may be practiced. These embodiments are described in sufficient detail to enable those skilled in the art to practice the invention, and it is to be understood that other embodiments may be utilized and that structural, logical and electrical changes may be made without departing from the scope of the present invention. The following description of example embodiments is, therefore, not to be taken in a limited sense, and the scope of the present invention is defined by the appended claims.

The functions or algorithms described herein may be implemented in software or a combination of software and human implemented procedures in one embodiment. The software may consist of computer executable instructions stored on computer readable media such as memory or other type of storage devices. Further, such functions correspond to modules, which are software, hardware, firmware or any combination thereof. Multiple functions may be performed in one or more modules as desired, and the embodiments described are merely examples. The software may be executed on a digital signal processor, ASIC, microprocessor, or other type of processor operating on a computer system, such as a personal computer, server or other computer system.

New generation hearing protection devices and communication systems will have sound exposure monitoring as an integrated feature. The typical use pattern of such products will vary between persons, but it seems likely that users will turn the equipment on and off several times during the day. This is in contrast to the way standard personal sound exposure meters (PSEM)s are being used, as they will be turned on as the work shift starts and then stay on during the entire shift. The reading of a standard PSEM at the end of the shift represents the sound exposure for that shift, not taking the HPD performance into account.

Health and Safety regulations typically set permissible daily sound exposure limits. The conventional interpretation of this is to estimate the sound exposure related to a given work shift, and compare this to limits given by regulations. One problem recognized by the inventor regards determining when a current work shift started, given that the unit may have been switched on and off during the day. Note that in any case, the assumption is that the user was not exposed to noise during the periods when the unit was switched off, at least not to a degree that will significantly influence the daily sound exposure.

In one embodiment, sound exposure is calculated in a sliding time window. This approach is simple for the user (requires no intervention) and safe (because the resulting exposure value represents a conservative estimate of what is achieved with the conventional/manual procedure).

The sliding window method re-defines the exposure at any time to be the cumulative exposure during the latest T hours. The window length T should be at least the duration of the longest shift that the worker could work, for the resulting exposure measure to be a safe estimate. The longer the window length T, the more conservative will the exposure estimate be. In one example work environment, involving an offshore oil & gas installations, the longest allowable work shifts are 16 hours, and T=16 hours may be used initially. The window may be adjusted to different values. Some non-limiting examples include 4 hours for a half shift, 8 hours, 12 hours and others.

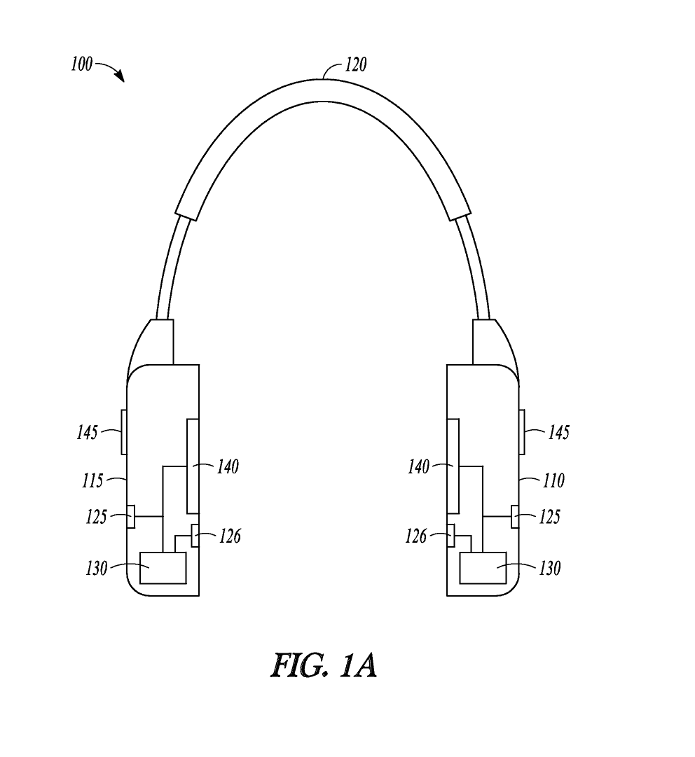

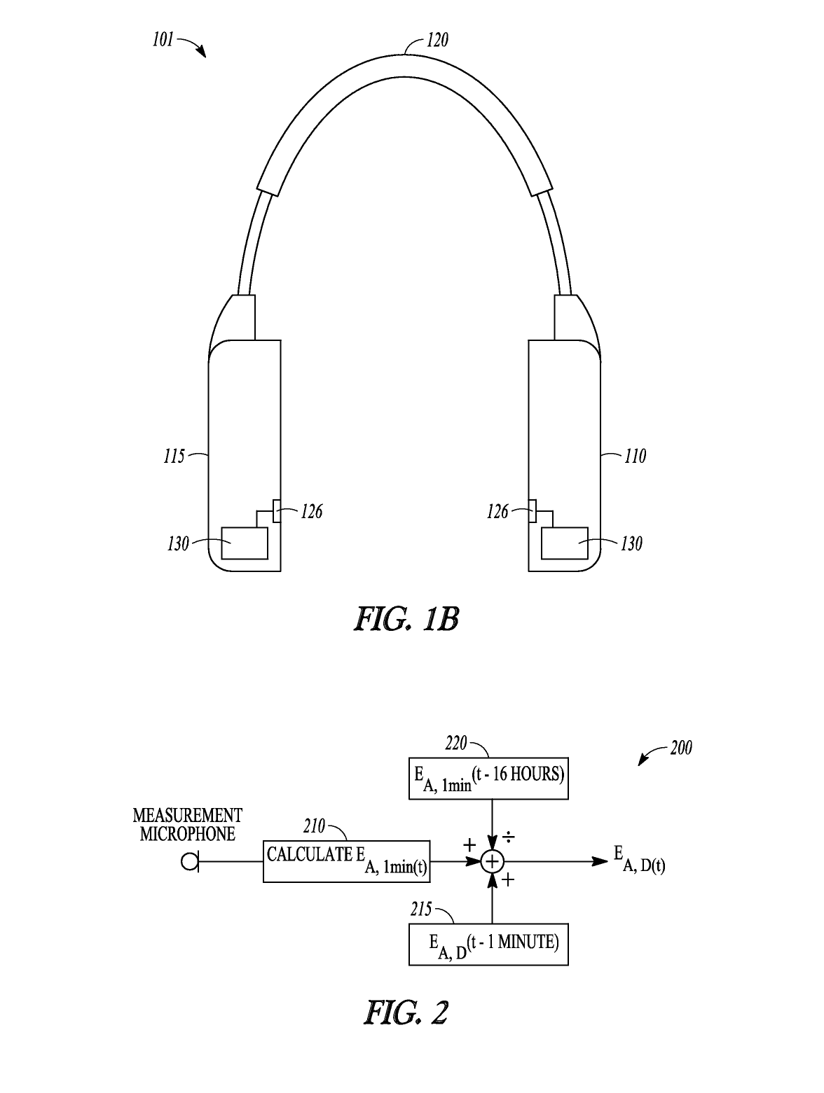

FIGS. 1A and 1B are block diagrams of an example hearing protection device 100 and 101 with integrated sound exposure monitoring. Protection device 100 is an active noise cancelling hearing protection device, while protection device 101 in FIG. 1B is a passive hearing protection device. Device 101 is shown with similar elements sharing the same reference numbers with device 100.

In one embodiment, two ear muffs that fit either over the ear or around the ear are illustrated at 110 and 115 connected by an adjustable band 120. In some embodiments, the hearing protection device may be in the form of cylindrical, bullet-shaped, or flanged earplugs for insertion into the ear as illustrated in later figures.

Referring to FIG. 1A, a microphone 125 is positioned on ear piece 110 to measure ambient noise. Microphone 125 in one embodiment is positioned on an outside portion of the ear piece. A separate microphone 126 is located inside the earpiece and is positioned to measure sound that the ear is exposed to after noise cancellation is performed. The measured noise is converted to digital signals and provided to a processor 130. Processor 130 performs noise cancellation calculations and provides a noise cancelling signal to a speaker 135 positioned to transmit cancelling noise to a speaker 140 positioned to transmit cancelling noise into the ear. Earpiece 115 may include the same microphone, processor, and speaker such that cancelling noise is provided to both ears. In some embodiments, a single processor in one of the earpieces, or in a separate wire or wireless connected external controller with user interface control buttons may provide the processing for noise cancellation.

In one embodiment, the sound captured by the outer microphone 125 is converted to digital signals and provided to a processor 130. The processor may filter the external sound detected by the outer microphone 125 (by filtering the signal to ensure that sounds are only reproduced at a safe level) and direct the speaker 130 to generate the filtered sounds within the user's ear (thereby allowing hear-through capabilities).

In a different embodiment, the processor 130 may receive signals representative of sound from an external audio source, such as a communication radio device or music listening system. The processor may filter the received signal and direct the speaker 130 to generate the sounds within the user's ear. The sounds may also be filtered to ensure that sounds are only reproduced at a safe level.

Processor 130 also performs the sliding window algorithm for both devices 100 and 101. Note that device 101 is passive, and that the processor need not perform active noise cancellation calculations, hear-through calculations or audio input calculations in device 101.

The sliding window algorithm may be performed in one earpiece in one embodiment, or both earpieces in further embodiments. Performing noise measurements using the sliding window algorithm separately for each ear may be used to ensure each ear is adequately protected from overexposure to noise. In further embodiments, a separate processor or other circuitry may be used to perform the sliding window algorithm. In one embodiment, at least one of the ear pieces or a separate controller includes a display, such as one or more light emitting diodes (LEDs) or other display 145 for providing a display of the sound exposure as calculated from the sliding window algorithm. In one embodiment, three LEDs that each emit one of red, yellow, and green light are used to represent high, medium and low exposure respectively, effectively providing an exposure indicator. The processor 130 may drive the display 145 in some embodiments. In further embodiments, the processor may generate speech to the user indicating exposure, and may further provide electronic signals representative of exposure.

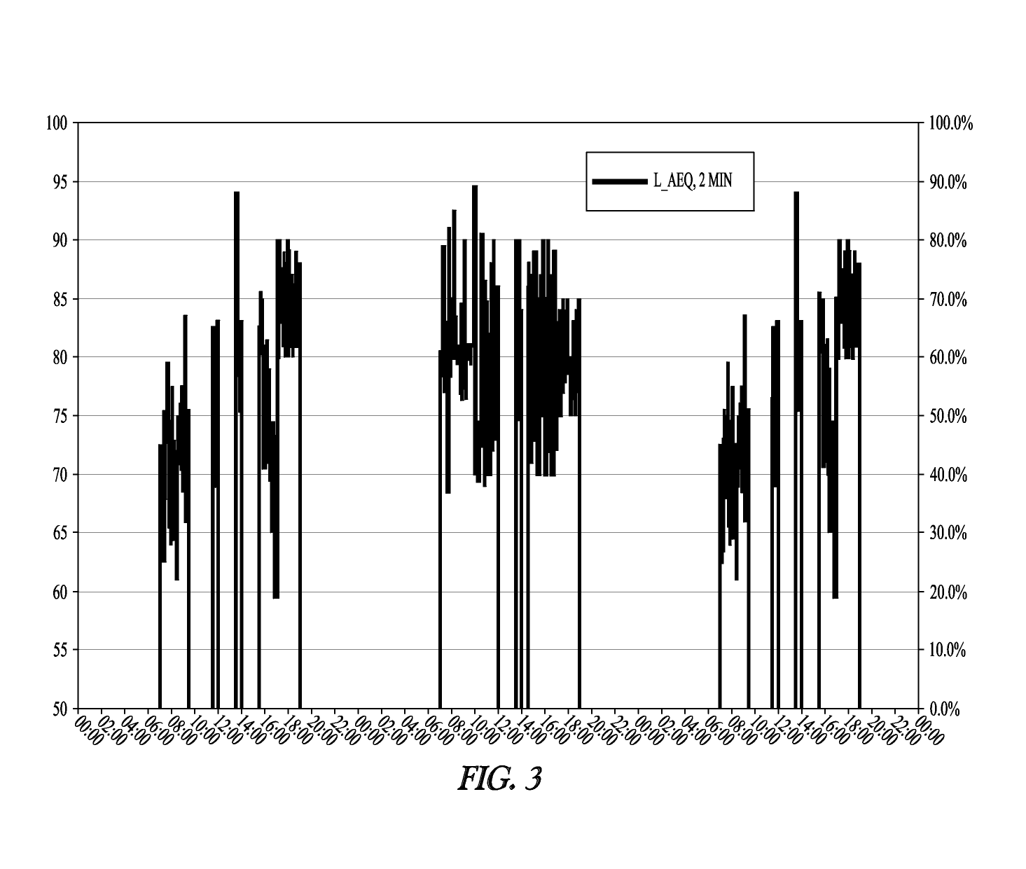

In one embodiment, operation of the sliding window is illustrated in block schematic form at 200 in FIG. 2. Based on the signal from the measurement microphones 126, the short duration A-weighted sound exposure is calculated at 210. Short duration typically means on the order of 1-2 minutes, and for the remainder of this explanation it is assumed to be 2 minutes. When this is calculated for the current time t, it is denoted E_{A,2min}(t). Every minute, the daily exposure estimate E_{A,D}(t) is updated by adding the recent short duration exposure value and subtracting the short duration value from T hours ago at 220. The short duration in various embodiments may vary between 30 seconds or less and two minutes, 10 seconds or less and 2 minutes, or between 1 and 3 minutes. Other durations may also be used consistent with a goal of minimizing risks of overexposure of a user to noise by not using too long a duration, while ensuring sufficient processing and memory capabilities to handle shorter durations.

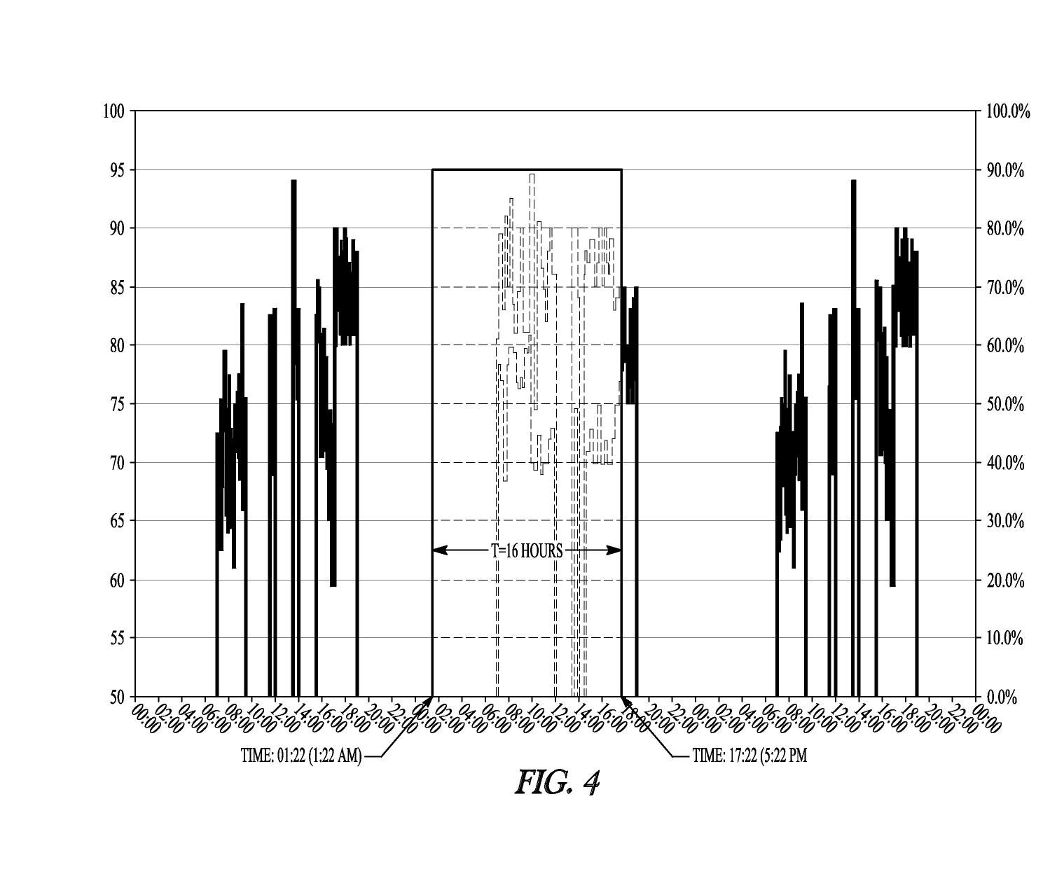

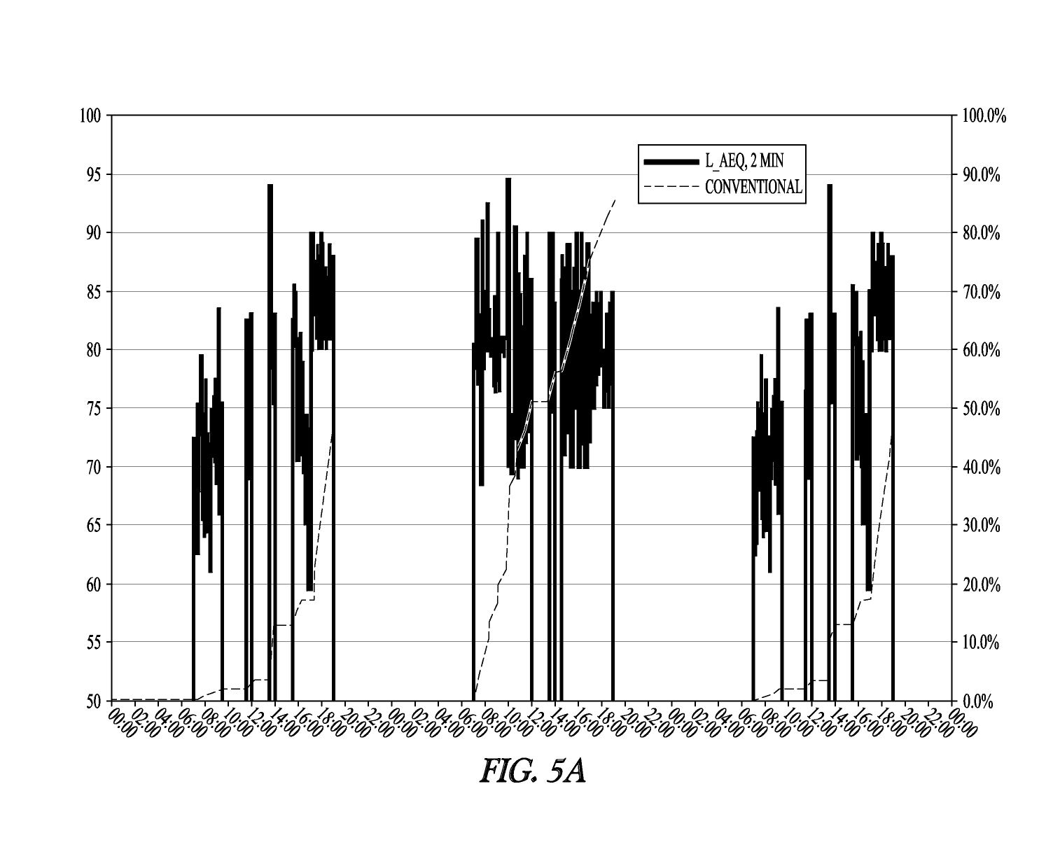

FIG. 3 shows example data illustrating sound exposure as a function of time. In principle these data could have been collected using any kind of PSEM, but may also be used in a hearing protection device with integrated PSEM capabilities. In FIG. 3, sound exposure is illustrated as an equivalent continuous A-weighted sound pressure levels, L_{Aeq.2min} on the y-axis and time on the x-axis. Three consecutive days of sound exposure is illustrated. FIG. 4 illustrates how at a given time (17:22 on day 2) the latest T hours (16 hours in this example) of noise are used for calculation of the noise dose at that time.

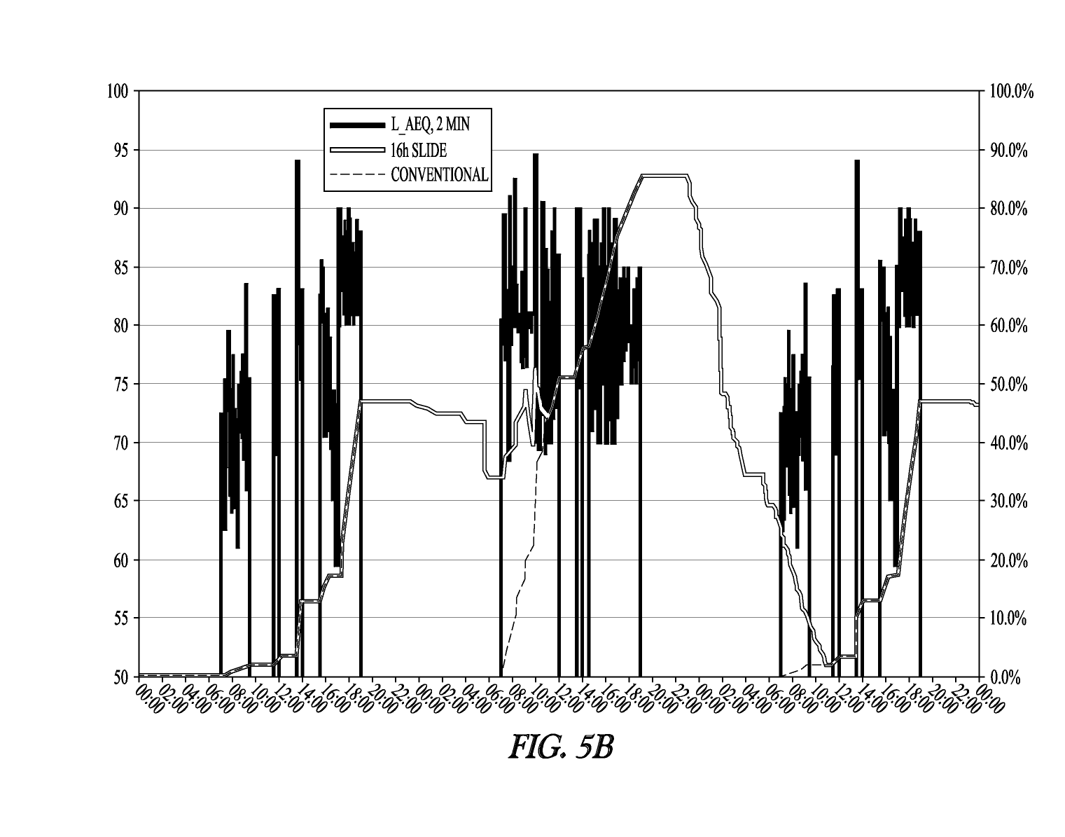

FIGS. 5A and 5B illustrate first the noise dose calculated according to the conventional approach and then by the sliding window method. In both cases noise dose is calculated as a percentage of a given exposure limit value and shown as a function of time. One example advantage with the sliding window algorithm is that it makes the operation very simple for the user. The user does not have to reset the dose to zero at any time, nor is there a need to configure the unit for different work patterns like day shift or night shift.

The sliding window method results in safe exposure estimates, as the estimate is bounded downwards by the conventional exposure estimate. The sliding window method to a much larger degree than the conventional approach resembles the mechanisms in the ear. The duration of the rest period is believed to have an impact on the likelihood of noise induced hearing loss. This is disregarded by the conventional exposure calculation approach because the dose is reset to zero at the beginning of every shift no matter how short the rest has been. With the sliding window approach a short rest period will also lead to increased exposure estimates for the work shift following after the rest period, and the exposure limit value could be reached sooner than with the conventional approach.

The way users will most likely notice the difference between the two approaches, is that while dose measured according to the conventional approach is monotonically increasing within each work shift, this is not true when the sliding window is being used. With the sliding window the dose could well go downwards even if the user is working in a moderately noisy environment. This means that the noise dose could go from a yellow zone to a green zone (the user is informed about sound exposure status by green, yellow and red indicator lights), or from a red zone to yellow zone during the work shift.

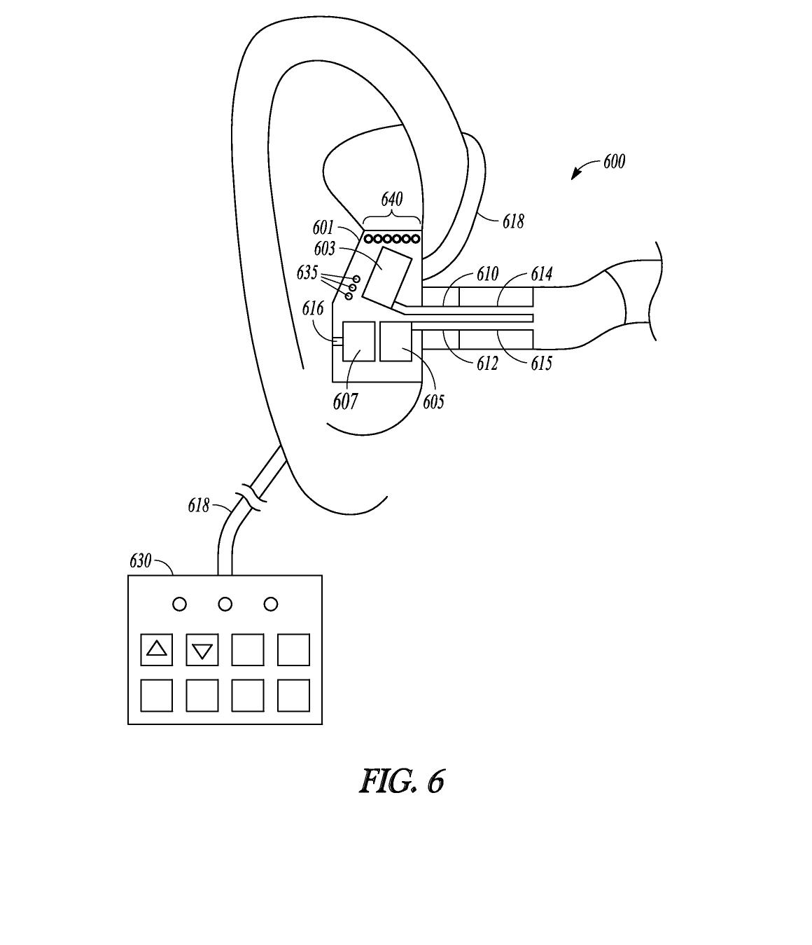

In one embodiment illustrated in FIG. 6, an earpiece 600 may be used with an external electronics unit. An earpiece housing 601 contains a loudspeaker 603, an inner microphone 605, and an outer microphone 607. Extending from the housing is an insert portion 610, which is designed to be inserted into an ear canal. The insert portion 610 includes a protective seal or sealing element 612 that forms a secure fit within the user's ear canal to passively block sound infiltration into the user's eardrum, serving as a passive attenuation hearing protection device. Additionally, there is a sound tube 614 that leads from the speaker's face, through the sealing section of the insert portion, and to the ear canal. Sound tube 614 directs the sound stimulus produced by the speaker 603 into the user's ear canal (so that it is incident upon the user's eardrum). Sound tube 615 leads from the inner microphone's face 605, through the sealing section of the insert portion, and to the ear canal. Sound tube 615 allows for the inner microphone 605 to detect noise levels within the user's ear canal (i.e. the sound incident upon the user's eardrum). In other words, the microphone might be directed towards the meatus. Sound tube 616 leads from the outer microphone 607, through the housing 601, to open to the outside world, allowing the outer microphone 607 to detect external sound. Sound tubes may be optional, as in some embodiments the microphone and/or speaker elements can be mounted directly on the appropriate face of the device.

Wire 618 connects the earpiece to an external electronics unit 630. The external electronics unit would typically include a user interface, storage memory with the required algorithms, including the sliding window algorithms. The electronics unit 630 may filter the external sound detected by the outer microphone (by filtering the signal to ensure that sounds are only reproduced at a safe level) and direct the speaker 603 to generate the filtered sounds within the user's ear (thereby allowing hear-through capabilities). The electronics unit 630 may be configured to assess sound exposure based on the signal from the inner microphone 605, and store sound exposure data on memory. And the electronics unit might also optionally have an interface for uploading of information from the memory/storage to an external computer system, or in some embodiments, operate red, yellow, and green exposure lights at 635, as well as provide audio alerts to users regarding sound exposure status. Various audio alerts may be provided as the wearer approaches different levels of sound exposure as calculated using the sliding window algorithm, such as transitions between low and medium and between medium and high exposures in each ear.

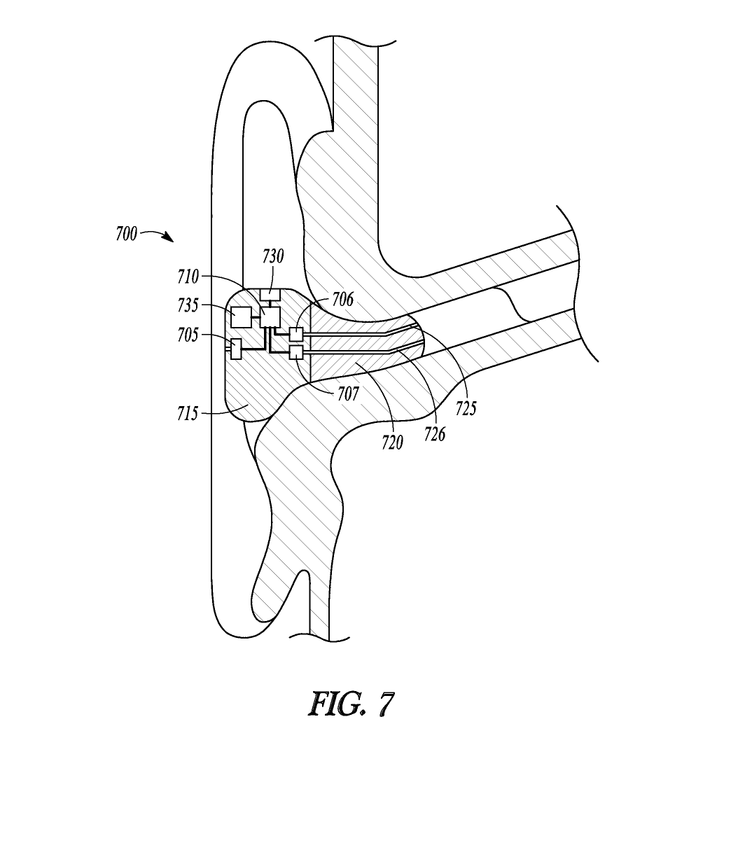

FIG. 7 is an illustration of a complete all-in-ear device 700 with capabilities for passive sound attenuation, active sound attenuation, sound exposure monitoring, leakage control, hear-through, and communication/entertainment, featuring passive sealing, electro-acoustic transducers, and electric circuitry. Device 700 has an outer section arranged for sitting adjacent to the outward facing portion of the sealing section and a part of the inward facing portion of the outer section is formed to fit the concha around the outer portion of the meatus. External sounds are attenuated by the sealing section (typically in the form of an earplug), inserted into the outer part of the ear canal or meatus. Optionally, external sounds may also be attenuated using active noise control, which is achieved by using one or two microphones M1 705 (an outer microphone), M2 706 (an inner microphone) and a loudspeaker SG 707 together with electronic circuits in an electronics unit 710 mounted in the device 700. Algorithms for active noise control are generally known and thus will not be described in detail herein, but may include active noise cancelling feedback of acoustic signals converted by at least one of the microphones through the loudspeaker SG 707.

Device 700 includes a main section 715 containing the two microphones M1 705, M2 706 and a loudspeaker SG 707. The main section 715 is designed to provide comfortable and secure placement in the concha. A sealing section 720 is attached to the main section. The sealing section 720 may be an integral part of the ear terminal, or it may be removable/interchangeable. The sound inlet of the outer microphone M1 705 is connected to the outside of the ear terminal, picking up external sounds. Inner microphone M2 706 is connected to the inner portion of the acoustic meatus by means of an acoustic transmission channel T1 725. The sound outlet of the loudspeaker SG 707 is open to the inner portion of the acoustic meatus by means of another acoustic transmission channel T2 726 between the loudspeaker SG and the inward facing portion of the sealing section. When smaller microphones and speakers are available, microphone M2 706 and speaker SG 707 may be mounted directly at the innermost part of the sealing section 720, such that there would be no need for transmission channels.

The two microphones and the loudspeaker are connected to electronics unit 710, which may optionally be connected to other equipment by an interface 730 that may transmit digital and/or analog signals, and also possibly power. An electronics module and optionally a power supply 735 (such as a battery) may be included in main section or in a separate section. The main section of the device 700 may be made of standard polymer materials of the sort that are used for hearing aids, for example. The sealing section may be made of a resilient, slowly re-expanding shape retaining polymer foam like PVC, PUR or other materials suitable for earplugs and other hearing protection devices. The channels may also be made of polymer wall material (or some other non-conforming material) in order to prevent their collapse when the sealing section is inserted into the meatus. When configured as a simple passive hearing protection device, only the inner microphone need be included, as no active noise cancellation, hear-through or external audio sources are utilized.

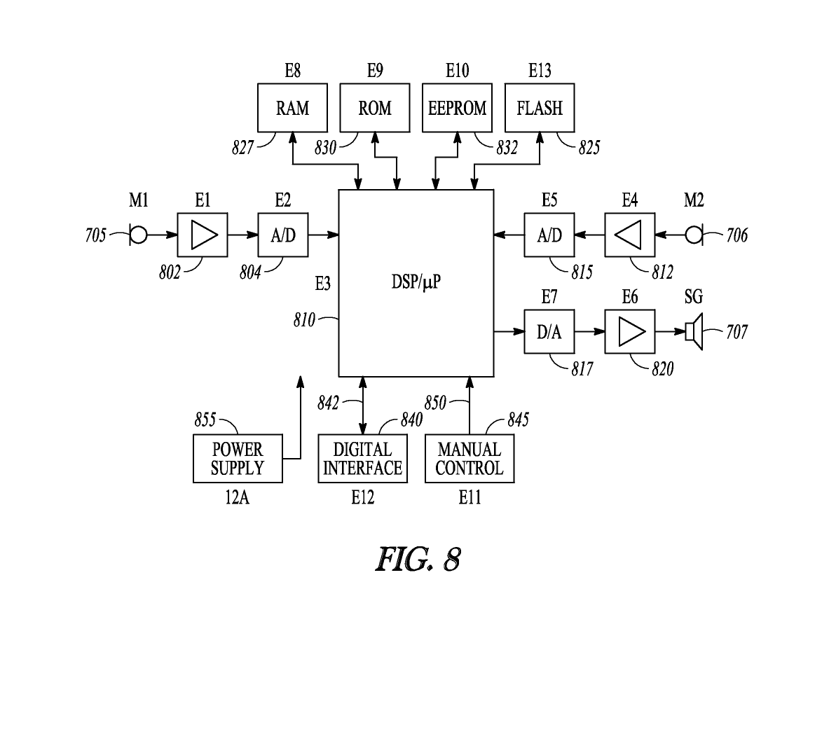

The electronics unit 710 may comprise electric circuitry as shown in FIG. 8, which may be configured and/or programmed to achieve several possible functions. By way of example, in an embodiment the outer microphone M1 705 may pick up ambient (external) sound. A signal from the outer microphone M1 705 may be amplified in and amplifier E1 802 and sampled and digitized in an analog-to-digital converter E2 804 and then fed to a processing unit E3 810 that may be a digital signal processor (DSP), a microprocessor, or a combination of the two. A signal from the inner microphone M2 706, which picks up sound in the meatus between the sealing section and the eardrum, may be amplified in amplifier E4 812 and sampled and digitized in the analog-to-digital converter E5 815 and fed to the processing unit E3 810. A desired digital signal is generated in the processing unit E3 810. This signal is converted to analog form in the digital-to-analog converter E7 817 and fed to the analog output amplifier E6 820 that drives the loudspeaker SG 707. The sound signal produced by the loudspeaker SG 707 is fed to the eardrum (tympanum) via channel T2 726 as described above.

The processing unit E3 810 in this embodiment is connected to memory elements such as flash memory E13 825, RAM (random access memory) E8 827, ROM (read only memory) E9 830, and EEPROM (electronically erasable programmable read only memory) E10 832. The memories or computer readable storage devices E8, E9, E10, and E13 are used for storing computer programs used to cause a processor 810 to perform algorithms such as noise cancellation and sound exposure calculations. The storage devices may also store one or more of, filter coefficients, test responses, test results, sound exposure data, analysis data, and/or other relevant data. The electronic circuitry may be connected to other electrical units via interface E12 840 (which may be via cable or wireless through a digital radio link represented at 842, such as Bluetooth standard). A manual control signal may be generated in E11 845 and fed to the processing unit E3 810 via connection 850. The control signal may be generated using a user interface with buttons, switches, etc. and may be used to turn the unit on and off, to change operation mode, to signal responses, etc. Buttons as shown at 640 in FIG. 6 may be individually assigned to generate these control signals, or may control one or more different functions depending on different modes of operation. In an alternative embodiment, a predetermined voice signal may serve as one or more control signals. The electric circuitry is powered by power supply 12A 855 that may be a primary or rechargeable battery arranged in the ear terminal or in a separate unit, or may be an electrical power connection.

### EXAMPLES

1. A system comprising:
a hearing protection device;
a first microphone coupled to the hearing protection device to sense sound between the hearing protection device and the eardrum; and
a processor coupled to receive signals from the first microphone representative of the sensed sound and to calculate sound exposure to the ear of the wearer over a sliding window of time duration.

2. The system of example 1 wherein the sliding window of time has a duration consistent with a longest allowable work shift of a wearer.

3. The system of example 1 and further comprising at least one exposure indicator to provide an indication to the wearer regarding calculated sound exposure.

4. The system of example 3 wherein the exposure indicator comprises at least one light emitting device.

5. The system of example 1 wherein sound exposure is calculated and summed for each of consecutive periods of time that are less than the sliding window of time, and wherein such periods older than the duration of the sliding window of time are subtracted from the sum.

6. The system of example 5 wherein the consecutive periods of time are equal and have a duration of between 30 seconds and 2 minutes.

7. The system of example 1 and further comprising:
a second microphone coupled to the hearing protection device to sense sound ambient to a wearer of the hearing protection device;
a loudspeaker coupled to the hearing protection device to transmit sound from the hearing protection device to the ear of the wearer of the hearing protection device; and
wherein the processor receives signals representative of sound from the first and/or second microphone and provides noise cancellation signals to the loudspeaker.

8. The system of example 1 and further comprising:
a second microphone coupled to the hearing protection device to sense sound ambient to a wearer of the hearing protection device;
a loudspeaker coupled to the hearing protection device to transmit sound from the hearing protection device to the ear of the wearer of the hearing protection device; and
wherein the processor receives signals representative of sound from the second microphone and provides signals representing the ambient sound to the loudspeaker.

9. The system of example 1 and further comprising:
an audio source connected to the hearing protection device to provide communication or entertainment signals;
a loudspeaker coupled to the hearing protection device to transmit sound from the hearing protection device to the ear of the wearer of the hearing protection device; and
wherein the processor receives signals from the audio source and provides signals representing the audio source to the loudspeaker.

10. The system of example 1 wherein the earpiece is adapted for insertion into an ear canal of the wearer, and wherein the processor is integrated into the earpiece.

11. The system of example 10 wherein the earpiece has a first canal extending from the speaker into the ear canal and a second canal extending from the ear canal to the second microphone.

12. A method comprising:
sensing sound on the inside of the protective seal of the hearing protection device when worn; and
using a sliding window algorithm to calculate sound to which the ear of the wearer is exposed.

13. The method of example 12 wherein the sliding window of time has a duration consistent with a longest allowable work shift of a wearer.

14. The method of example 12 and further comprising providing an indication to the wearer regarding calculated sound exposure.

15. The method of example 14 wherein the exposure indicator is visible light of different colors.

16. The method of example 14 wherein the exposure indicator comprises an audible sound.

17. The method of example 12 wherein sound exposure is calculated and summed for each of consecutive periods of time that are less than the sliding window of time, and wherein such periods older than the duration of the sliding window of time are subtracted from the sum.

18. The method of example 12 and further comprising:
sensing ambient sound in an environment of the wearer of the hearing protection device and/or sound from the inside of the protective seal of the hearing protection device; and
generating sound to cancel the sensed ambient sound within the ear of the wearer.

19. The method of example 12 and further comprising:
sensing ambient sound in an environment of the wearer of the hearing protection device; and
generating sound to represent the sensed ambient sound within the ear of the wearer.

20. The method of example 12 and further comprising:
receiving communication or entertainment sound from an external audio source; and
generating sound to represent the communication or entertainment sound within the ear of the wearer.

21. A computer readable storage device having stored instructions to cause a computer system to execute a method, the method comprising:
sensing sound within the ear of a wearer of a hearing protection device; and
using a sliding window algorithm to calculate sound to which the ear of the wearer is exposed.

22. The computer readable storage device of example 21 wherein the sliding window of time has a duration consistent with a longest allowable work shift of a wearer, and wherein the method further includes providing an indication to the wearer regarding calculated sound exposure.

23. The computer readable storage device of example 21 wherein sound exposure is calculated and summed for each of consecutive equal periods of time that are less than the sliding window of time, and wherein such periods older than the duration of the sliding window of time are subtracted from the sum.

24. The computer readable storage device of example 21 wherein the method further comprises:
sensing ambient sound in an environment of the wearer of the hearing protection device and/or sound from the inside of the protective seal of the hearing protection device; and
generating sound to cancel the sensed ambient sound within the ear of the wearer.

25. The computer readable storage device of example 21 wherein the method further comprises:
sensing ambient sound in an environment of the wearer of the hearing protection device; and
generating sound to represent the sensed ambient sound within the ear of the wearer.

26. The computer readable storage device of example 21 wherein the method further comprises:
receiving communication or entertainment sound from an external audio source; and
generating sound to represent the communication or entertainment sound within the ear of the wearer.

Although a few embodiments have been described in detail above, other modifications are possible. For example, the logic flows depicted in the figures do not require the particular order shown, or sequential order, to achieve desirable results. Other steps may be provided, or steps may be eliminated, from the described flows, and other components may be added to, or removed from, the described systems. Other embodiments may be within the scope of the following claims.

1. A system comprising:
a hearing protection device;
a first microphone coupled to the hearing protection device to sense sound between the hearing protection device and the eardrum; and
a processor coupled to receive signals from the first microphone representative of the sensed sound and to calculate sound exposure to the ear of the wearer over a sliding window of time duration.

2. The system of claim 1 wherein the sliding window of time has a duration consistent with a longest allowable work shift of a wearer.

3. The system of claim 1 and further comprising at least one exposure indicator to provide an indication to the wearer regarding calculated sound exposure.

4. The system of claim 3 wherein the exposure indicator comprises at least one light emitting device.

5. The system of claim 1 wherein sound exposure is calculated and summed for each of consecutive periods of time that are less than the sliding window of time, and wherein such periods older than the duration of the sliding window of time are subtracted from the sum.

6. The system of claim 5 wherein the consecutive periods of time are equal and have a duration of between 30 seconds and 2 minutes.

7. The system of claim 1 and further comprising:
a second microphone coupled to the hearing protection device to sense sound ambient to a wearer of the hearing protection device;
a loudspeaker coupled to the hearing protection device to transmit sound from the hearing protection device to the ear of the wearer of the hearing protection device; and
wherein the processor receives signals representative of sound from the first and/or second microphone and provides noise cancellation signals to the loudspeaker.

8. The system of claim 1 and further comprising:
a second microphone coupled to the hearing protection device to sense sound ambient to a wearer of the hearing protection device;
a loudspeaker coupled to the hearing protection device to transmit sound from the hearing protection device to the ear of the wearer of the hearing protection device; and
wherein the processor receives signals representative of sound from the second microphone and provides signals representing the ambient sound to the loudspeaker.

9. The system of claim 1 and further comprising:
an audio source connected to the hearing protection device to provide communication or entertainment signals;
a loudspeaker coupled to the hearing protection device to transmit sound from the hearing protection device to the ear of the wearer of the hearing protection device; and
wherein the processor receives signals from the audio source and provides signals representing the audio source to the loudspeaker.

10. The system of claim 1 wherein the earpiece is adapted for insertion into an ear canal of the wearer, and wherein the processor is integrated into the earpiece.

11. The system of claim 10 wherein the earpiece has a first canal extending from the speaker into the ear canal and a second canal extending from the ear canal to the second microphone.

12. A method comprising:
sensing sound on the inside of the protective seal of the hearing protection device when worn; and
using a sliding window algorithm to calculate sound to which the ear of the wearer is exposed.

13. The method of claim 12 wherein the sliding window of time has a duration consistent with a longest allowable work shift of a wearer.

14. The method of claim 12 and further comprising providing an indication to the wearer regarding calculated sound exposure.

15. The method of claim 14 wherein the exposure indicator is visible light of different colors.

16. The method of claim 14 wherein the exposure indicator comprises an audible sound.

17. The method of claim 12 wherein sound exposure is calculated and summed for each of consecutive periods of time that are less than the sliding window of time, and wherein such periods older than the duration of the sliding window of time are subtracted from the sum.

18. The method of claim 12 and further comprising:
sensing ambient sound in an environment of the wearer of the hearing protection device and/or sound from the inside of the protective seal of the hearing protection device; and
generating sound to cancel the sensed ambient sound within the ear of the wearer.

19. The method of claim 12 and further comprising:
sensing ambient sound in an environment of the wearer of the hearing protection device; and
generating sound to represent the sensed ambient sound within the ear of the wearer.

20. The method of claim 12 and further comprising:
receiving communication or entertainment sound from an external audio source; and
generating sound to represent the communication or entertainment sound within the ear of the wearer.

21. A computer readable storage device having stored instructions to cause a computer system to execute a method, the method comprising:
sensing sound within the ear of a wearer of a hearing protection device; and
using a sliding window algorithm to calculate sound to which the ear of the wearer is exposed.

22. The computer readable storage device of claim 21 wherein the sliding window of time has a duration consistent with a longest allowable work shift of a wearer, and wherein the method further includes providing an indication to the wearer regarding calculated sound exposure.

23. The computer readable storage device of claim 21 wherein sound exposure is calculated and summed for each of consecutive equal periods of time that are less than the sliding window of time, and wherein such periods older than the duration of the sliding window of time are subtracted from the sum.

24. The computer readable storage device of claim 21 wherein the method further comprises:
sensing a
\mbient sound in an environment of the wearer of the hearing protection device and/or sound from the inside of the protective seal of the hearing protection device; and
generating sound to cancel the sensed ambient sound within the ear of the wearer.

25. The computer readable storage device of claim 21 wherein the method further comprises:
sensing ambient sound in an environment of the wearer of the hearing protection device; and
generating sound to represent the sensed ambient sound within the ear of the wearer.

26. The computer readable storage device of claim 21 wherein the method further comprises:
receiving communication or entertainment sound from an external audio source; and
generating sound to represent the communication or entertainment sound within the ear of the wearer.
